(19) United States
(12) Patent Application Publication
(10) Pub. No.: US 2010/0004338 A1
(43) Pub. Date: Jan. 7, 2010

(54) TOPICAL GEL COMPOSITION COMPRISING AZELAIC ACID

(75) Inventors: Nilendu Sen, Mumbai (IN); Olympia Noronha, Rajasthan (IN)

Correspondence Address: GLENMARK PHARMACEUTICALS INC USA, 750 CORPORATE DRIVE, MAHWAH, NJ, 07430, US

(73) Assignee: GLENMARK GENERICS LTD., Mumbai (IN)

(21) Appl. No.: 12/494,353

(22) Filed: Jun. 30, 2009

Related U.S. Application Data
(60) Provisional application No. 61/163,077, filed on Mar. 25, 2009.

(30) Foreign Application Priority Data
Jul. 3, 2008 (IN) 1383/MUM/2008

(51) Int. Cl. A61K 31/194; C07C 55/18; A61P 17/10

(52) U.S. Cl. 514/574; 562/590

(57) ABSTRACT

The present invention discloses a novel azelaic acid topical pharmaceutical gel composition that does not contain lecithin and a process for its preparation.

### PRIORITY

This application claims the benefit to Indian Provisional Application 1383/MUM/2008, filed on Jul. 3, 2008, and under 35 U.S.C. §119 to U.S. Provisional Application 61/163,077, filed Mar. 25, 2009, the contents of each of which, are incorporated by reference herein.

### BACKGROUND OF THE INVENTION

1. Technical Field

The present invention relates to a pharmaceutical gel composition for topical delivery comprising a pharmaceutically effective amount of azelaic acid and a process for its preparation.

2. Description of the Related Art

Azelaic acid is a saturated dicarboxylic acid found naturally in wheat, rye, and barley. It is a natural substance that is produced by Malassezia furfur (also known as Pityrosporum ovale), yeast that lives on normal skin. It is effective against a number of skin conditions, such as mild to moderate acne, when applied topically in a cream formulation of 20%. Azelaic acid may be useful as a hair growth stimulant.

Azelaic acid has the molecular structure, as shown below, with a molecular formula, C₉H₁₆O₄ and molecular weight 188.22.

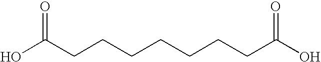

U.S. Pat. No. 4,713,394 discloses a composition containing azelaic acid, useful for the treatment of non-acne inflammatory dermatoses, infectious cutaneous diseases, and hair loss resulting from inflammation or hormonal anomalies.

U.S. Pat. No. 6,534,070 relates to a hydrogel composition comprising azelaic acid, triacylglyceride, propylene glycol and polysorbate, in an aqueous phase that further comprises water and salts, The disclosed composition contains polyacrylic acid and lecithin.

### SUMMARY OF THE INVENTION

The present invention provides a gel comprising azelaic acid and at least one pharmaceutically acceptable carrier.

The present invention provides a gel comprising azelaic acid and at least one pharmaceutically acceptable carrier, wherein the formulation does not include lecithin.

The present invention provides a topical gel comprising azelaic acid in a monophase or multiphase system.

The present invention provides a topical gel comprising azelaic acid, wherein the gel is oil in water type emulsion gel.

The present invention provides a topical gel comprising azelaic acid, wherein the gel is oil in water type emulsion gel, wherein the formulation does not contain lecithin.

The present invention provides a topical gel comprising azelaic acid, wherein the gel is in single phase.

The present invention provides a topical gel comprising azelaic acid, wherein the gel is in single phase, wherein the formulation does not contain lecithin.

The present invention provides a topical gel comprising azelaic acid for the treatment of rosacea, presbyderma, melasma, acne and/or skin irritations, wherein the formulation does not contain lecithin.

### DETAILED DESCRIPTION OF THE INVENTION

The present invention discloses a novel azelaic acid topical pharmaceutical gel composition that does not contain lecithin, and a process for its preparation.

Administration of physiologically active agents through the skin (‘topical drug delivery’) provides a relatively slow and controlled route for release of a physiologically active agent into the local tissue. Topically administered azelaic acid (FINACEA® Gel) 15%, is indicated for topical treatment of inflammatory papules and pustules of mild to moderate rosacea. However, topical drug delivery is complicated by the fact that the skin behaves as a natural barrier and therefore transport of agents through the skin is a complex mechanism.

For effective local delivery of a physiologically active agent that is applied to the surface of the skin (topical application), the agent must be partitioned firstly from the vehicle into the stratum corneum, it must typically then be diffused within the stratum corneum before being partitioned from the stratum corneum to the local tissues including the viable epidermis, dermis, subcutis and appendageal.

To overcome some of the problems with topical delivery that are associated with transport across the dermal layers (percutaneous absorption), physiologically active agents are commonly formulated in such a way that they are either in solubilized or dispersed in the final dosage form to allow passage through the skin barriers such that a complete therapeutic dose can be made available for the action.

Topical route offers continuity of drug administration, permits use of therapeutic agents with short biological half-lives, provides treatment of cutaneous manifestations of diseases usually treated systemically delivers medication directly into the systemic circulation and fosters ease of use and total patient compliance.

Surprisingly the gel formulation of azelaic acid without lecithin, described herein, in a resultant product is physicochemically stable and has desired therapeutic effect.

The present invention relates to a topical gel composition of azelaic acid, without lecithin and the composition according to the invention has the advantage that it allows a larger amount of pharmaceutical active ingredient to penetrate into living skin layers and/or cutaneous organs. The composition of present invention is suited for the treatment of rosacea, presbyderma, melasma or skin irritations.

The present invention provides a pharmaceutical gel composition comprising azelaic acid, in a monophase or multiphase system that does not contain lecithin.

The term gel, as herein used, is intended to mean a pharmaceutical composition comprising a pharmaceutically active ingredient either in single phase or in multiphase system; and the multiphase system having, one oil phase and one aqueous phase, wherein the oil phase is dispersed uniformly in aqueous phase.

The gel composition further comprises one or more surfactants, a co-solvent or oil, gellant, an alkalizer and a vehicle. Ingredients, which are suitable to form a gel and known to one skilled in the art can also be used to prepare the gel composition herein described.

The formulation may also contain one or more emulsifiers. Emulsifiers in a gel formulation are generally nonionic, anionic, cationic or amphoteric surfactants. Suitable emulsifiers include, but are not limited to, nonionic surface active agents, e.g., polysorbate (TWEEN®, EMULROR®, TRITON® and SIMULSOL® M-53), sorbitan monostearate, polyglyceryl-4 oleate, polyoxyethylene(4)lauryl ether or trivalent cationic and the like, and sodium lauryl sulphate and mixtures thereof. The preferred emulsifiers include polysorbate 80 or emollient surfactants like polypropylene glycol (PPG) stearyl ethers like PPG ethers of stearyl alcohol like PPG-20 methyl glucose ether distearate, PPG-15 Stearyl Ether and PPG-11 Stearyl Ether. More preferably PPG-20 methyl glucose ether distearate or mixtures thereof.

Suitable gelling agent for use in the invention may be selected from any of synthetic or semi-synthetic polymeric materials, polyacrylate copolymers, cellulose derivatives and polymethyl vinyl ether/maleic anhydride copolymers. Various carbomers such as Carbopol® 934, 940, 941, 974, 980, 981, 1342, 5984, ETD2020®, ETD2050®, Ultrez 10® (Manufacturer-Noveon) may be used herein. The preferred gelling agent is Carbopol®980. Carbomers are synthetic high molecular weight polymer of acrylic acid that are crosslinked with either allylsucrose or allylethers of pentaerythritol having a molecular weight of 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. In the present pharmaceutical formulations it is used as a suspending or viscosity-increasing agent. Aqueous solution of Carbopol® is acidic in nature due to the presence of free carboxylic acid residues. Neutralization of this solution cross-links and gelatinizes the polymer to form a viscous integral structure of desired viscosity.

Alkalizers or alkanizing agents include organic and inorganic basic compounds. Examples of inorganic basic salts that may be used in the present invention include ammonium hydroxide, alkali metal salts, and alkaline earth metal salts such as magnesium oxide, magnesium hydroxide, calcium hydroxide, sodium hydroxide, potassium hydroxide, lithium hydroxide, aluminum hydroxide, potassium carbonate, sodium bicarbonate and the like, preferably aqueous sodium hydroxide solution.

Vehicles are the substances that are generally used to make up the volume and can be polar or non-polar solvents or a mixture thereof. Example of a polar solvent is water, alcohol and the like. -Non-polar solvents include cyclohexane, carbon tetrachloride and the like. Other vehicles for use in this invention can be any other vehicle known to the person skilled in the art. The preferred vehicle(s) include water or alcohol, more preferably water.

The oil phase of the compositions described herein may contain at least one oil such as isohexadecane, polydecene and polyisobutene, fatty alcohols such as octyldodecanol, isostearyl alcohol and oleyl alcohol, esters such as essential fatty acid glycerides, triglycerides of capric and caprylic acids (Caprylic capric triglyceride (CCTG)), glyceryl monostearate, isopropyl myristate and mixtures thereof, and linear or branched fatty acid esters with fatty alcohols such as purcellin oil (stearyl octanoate). The preferred oil phase contsins isopropyl myristate.

Co-solvents which may be used herein include propylene glycols, polyethylene glycols, glyceryl monostearates and sorbitols, preferably propylene glycols.

The aqueous phase of the composition described herein may also contain various conventional additives. Among these include preservatives, fragrances, pigments (like TiO₂), and dyestuffs. Based on its additional antimicrobial feature, benzoic acid is the preferred preservative, while ethylene diaminetetraacetic acid (EDTA) is the preferred chelating agent.

The compositions described herein can further contain one or more additional excipients as known to the person skilled in the art that can be used to make azelaic acid gel composition in the form of a multiphase system.

Illustratively, benzoic acid and EDTA are dissolved in about 60 parts to about 70 parts of water. Then, a mixture of about 1 part of PPG-20 methyl glucose ether distearate (to form a single phase) or isopropyl myristate (to form a multiphase) and about 1.5 parts of polysorbate 80 is added and homogenized while being stirred. Propylene glycol is then added into the single phase system or multiphase system and homogenized. The resultant solution or pre-emulsion is then heated to about 50° C. and Carbopol® 980 is added under stirring, to which about 15 parts of azelaic acid is then added. Then, sodium hydroxide solution is added for gel formation. The resulting gel has approximately about four times higher availability of azelaic acid in living skin layers and/or cutaneous organs.

The examples, which follow, are not intended to be limiting of the scope of the present invention but read in conjunction with the detailed and general description above, provide further understanding of the present invention and an outline of a process for preparing the compositions of the invention.

### EXAMPLES

### Example 1

### Preparation of Pharmaceutical Gel for Topical Delivery is Given in Table 1

TABLE 1

| S. No. | Ingredients | % w/w |
|---|---|---|
| 1. | Azelaic acid | 15 |
| 2. | Benzoic acid | 0.1 |
| 3. | Disodium - EDTA | 0.1 |
| 4. | Carbopol ® 980 | 0.85 |
| 5. | Polysorbate 80 | 1.5 |
| 6. | Propylene glycol | 12 |
| 7. | PPG-20 methyl glucose ether distearate | 2.0 |
| 8. | Sodium hydroxide to adjust pH between 4.5 to 4.8 | |
| 9. | Purified water | 68.55 |

### Brief Manufacturing Procedure

1. Benzoic acid and EDTA are dissolved in 60 to 70 parts of water.
2. Then, a mixture of 2 parts of PPG-20 methyl glucose ether distearate and 1.5 parts of polysorbate 80 is added and homogenized while being stirred.
3. Propylene glycol is then added and homogenized.
4. The resultant mixture is then heated to 50° C. and Carbopol® 980 is added under stirring, Stirring is continued for 15 minutes until all of the Carbopol® 980 is uniformly dispersed.
5. Azelaic acid is introduced into 4). Sodium hydroxide solution is added for gel formation.

### Example 2

### Preparation of Pharmaceutical Gel for Topical Delivery is Given in Table 2

TABLE 2

| S. No. | Ingredients | % w/w |
|---|---|---|
| 1. | Azelaic acid | 15 |
| 2. | Benzoic acid | 0.1 |
| 3. | Disodium - EDTA | 0.1 |
| 4. | Carbopol ® 980 | 0.85 |
| 5. | Polysorbate 80 | 1.5 |
| 6. | Propylene glycol | 12 |
| 7. | Isopropyl myristate | 2.0 |
| 8. | Sodium hydroxide to adjust pH between 4.5 to 4.8 | |
| 9. | Purified water | 68.55 |

### Brief Manufacturing Procedure

1. Benzoic acid and EDTA are dissolved in 60 to 70 parts of water.
2. Then, a mixture of 2 parts of isopropyl myristate and 1.5 parts of polysorbate 80 is to be added and homogenized while being stirred.
3. Propylene glycol is then stirred into the pre-emulsion and homogenized.
4. The resultant pre-emulsion is then heated to 50° C. and Carbopol® 980 is added under stirring. Stirring is continued for 15 minutes until all of the Carbopol® 980 is uniformly dispersed.
5. Azelaic acid is introduced into 4). Then, sodium hydroxide solution is added for gel formation.

1. An azelaic acid gel composition, essentially free of lecithin.

2. The composition of claim 1, comprising azelaic acid from about 1 wt % to about 20 wt %.

3. The composition of claim 1, further comprising the azelaic acid, one or more surfactant, gellant, an alkalizer, vehicle and other auxiliary substances.

4. The composition of claim 3, wherein surfactant is selected from the group of polysorbates, sorbitan monostearate, polyglyceryl-4-oleate, polyoxyethylene(4)lauryl ether or trivalent cationics, and sodium lauryl sulphate or mixtures thereof.

5. The composition of claim 3, wherein the gellant is selected from the group of synthetic or semi-synthetic polymeric materials, polyacrylate polymers, cellulose derivatives and polymethylvinyl ether/maleic anhydride copolymers

6. The composition of claim 3, wherein the alkalizer is selected from magnesium oxide, magnesium hydroxide, calcium hydroxide, sodium hydroxide, potassium hydroxide, lithium hydroxide, aluminum hydroxide, potassium carbonate, sodium bicarbonate.

7. The composition of claim 3, wherein the vehicle is selected from water, alcohol, cyclohexane, carbon tetrachloride.

8. The composition of claim 3, wherein other auxiliary substances are selected from the group comprising of propylene glycols, polyethylene glycols, glyceryl monostearates and sorbitols, preferably propylene glycols, isopropyl myristate, glyceryl monostearate, isohexadecane, polydecene and polyisobutene, fatty alcohols, esters such as essential fatty acid glycerides, triglycerides of capric and caprylic acids (Caprylic capric triglyceride (CCTG)) or mixtures thereof.

9. A gel composition comprising of about 15 wt % azelaic acid; about 0.1 wt % benzoic acid; about 0.1 wt % disodium ethylenediaminetetraacetic acid; about 0.85 wt % Carbomer® 940 (Carbopol® 980); about 1.5 wt % Polysorbate® 80; about 12 wt % propylene glycol; about 2.0 wt % isopropyl myristate; about 0.2 wt % sodium hydroxide and purified water

10. The composition of claim 9 administered for the treatment of rosacea, presbyderma, melasma, acne and/or skin irritations.

11. A process for preparing an azelaic acid gel composition, comprising (a) forming a gel base by mixing a vehicle and an oily substance with a gelling agent and (b) addition of azelaic acid with stirring and then (c) addition of alkalizing agent to form the gel.

12. The process of claim 11, wherein the oily substance is isopropyl myristate, from about 0.5% to about 10%.

13. The process of claim 11, wherein the gelling agent is a polyacrylate polymer from about 0.01% to 2.0%.
